(19) United States
(12) Patent Application Publication
(10) Pub. No.: US 2025/0388557 A1
(43) Pub. Date: Dec. 25, 2025

(54) Method for Preparing High-purity Theaflavins by Coupling Macroporous Resin Columns

(71) Applicant: Jiangnan University, Wuxi (CN)

(72) Inventors: Hongxin Wang, Wuxi (CN); Yangjie Han, Wuxi (CN); Beiqi Wu, Wuxi (CN); Zaixiang Lou, Wuxi (CN); Chaoyang Ma, Wuxi (CN); Wenping Lv, Wuxi (CN)

(21) Appl. No.: 19/090,574

(22) Filed: Mar. 26, 2025

Related U.S. Application Data
(63) Continuation of application No. PCT/CN2024/121679, filed on Sep. 27, 2024.

(30) Foreign Application Priority Data
Jun. 25, 2024 (CN) 2024108265536

(51) Int. Cl. C07D 311/62; B01D 15/20; B01D 15/42

(52) U.S. Cl. CPC C07D 311/62; B01D 15/203; B01D 15/426

(57) ABSTRACT

Disclosed is a method for preparing high-purity theaflavins by coupling macroporous resin columns, belonging to the technical field of food engineering. A crude theaflavin solution with a purity of 15%-30% is loaded onto an LX-20B resin column and subjected to gradient elution according to a specific procedure; eluent is concentrated by rotary evaporation and/or freeze-dried to obtain a primary purified product; a solution of the primary purified product is loaded onto an AB-8 resin column and subjected to gradient elution according to the specific procedure; and eluent is concentrated by rotary evaporation and freeze-dried to obtain a high-purity theaflavins product with a purity of 95% or more, which can meet demands for high-purity theaflavins in some high-end health products and pharmaceutical research and development fields, and thus broadens an application range of theaflavins.

### TECHNICAL FIELD

The present disclosure belongs to the technical field of food engineering, and relates to a deep processing technology of natural products, in particular to a method for preparing high-purity theaflavins by coupling macroporous resin columns.

### BACKGROUND

Theaflavins (TFs) in tea have antioxidant, anti-tumor, antibacterial and antiviral, anti-inflammatory, blood lipid regulating, neuroprotection, and other various functions, and they are superior to catechins in certain functional activities. Therefore, theaflavins have a great commercial value, and prospects of theaflavins for application in industries such as food, medicine, and daily use chemical are also very broad. The content of theaflavins in natural tea leaves is relatively low, accounting for about 0.3%-1.5% of a dry weight of black tea. Moreover, at present, the purity of commercialized theaflavins is generally 20%-40%, which cannot meet demands for higher purity theaflavins in some high-end products and pharmaceutical research and development.

There are more than 20 types of theaflavin compounds in the current reports, among which theaflavin (TF1) in non-ester type theaflavin, and theaflavin-3-monogallate (TF2A), theaflavin-3′-monogallate (TF2B) and theaflavin-3, 3′-digallate (TF3) in ester type theaflavins are the most abundant, accounting for about 96% of the total TFs. The structures of the four major theaflavins are shown in FIG. 1, which are respectively formed by epicatechin (EC) and epigallocatechin (EGC), EC and epigallocatechin gallate (EGCG), EGC and epicatechin gallate (ECG), EGC and EGCG through oxidative coupling.

Because tea extracts and crude theaflavins contain a large number of other components and catechins with similar structures, it is a major technical problem to prepare high-purity theaflavins in the industry.

At present, the purification methods of theaflavins mainly include high-speed counter-current chromatography, medium-pressure preparation liquid phase method, gel chromatography, silica gel chromatography, cellulose chromatography, and the like. Such methods have disadvantages such as high cost, cumbersome operation, and high toxicity of organic reagents, and thus cannot realize the large-scale and commercial production of theaflavins. Macroporous resin has advantages of being large in adsorption capacity, fast in adsorption speed, good in selectivity, simple in regeneration treatment, low in resin cost, and the like. Macroporous resin method has also made some achievements in the separation and purification of theaflavins. According to reports, Yu Jun et al. used NKA-9 resin to increase the purity of theaflavins from 16.6% to 44.1%, with a yield of 56.67%. Yang Mingqi used HZ-818 resin to increase the purity of theaflavins from 20% to 53.6%, with a yield of 33.2%. Yuan Bin used HP-20 resin to increase the purity of theaflavins from 30.49% to 59.64%, with a yield of 20%. Liu Hongtao used HZ-816 resin to increase the purity of theaflavins from 52% to 90.8%, with a yield of 56%.

Up to now, no reports have been found that the purity of theaflavins purified by macroporous resin has increased from 15%-30% to 95% or more. The present disclosure uses macroporous resin column coupling to prepare high-purity theaflavins, with a purity reaching up to 95% or more, which lays a foundation for the research and development of high-end products using theaflavins as raw materials.

### SUMMARY

In the prior art, due to the similarity in structures of theaflavins and catechins, and the presence of a large number of other components in crude theaflavins, separation is difficult. Therefore, a purity of theaflavin products obtained by separation using macroporous resin is low, with little increase. In addition, other existing technologies have disadvantages such as cumbersome operation, high cost, and high toxicity, which cannot adapt to the development of large-scale mass separation and purification of theaflavins.

In order to overcome the shortcomings of the prior art, the purpose of the present disclosure is to provide a method for preparing high-purity theaflavins by coupling macroporous resin columns, which solves the problems of high cost, cumbersome operation, high toxicity and the like in the existing separation and purification technologies. A high-purity theaflavin product is obtained by employing macroporous resin and only using ethanol and water as eluents, and the yield of theaflavins is also improved. Furthermore, the macroporous resin columns can be used repeatedly, thus being suitable for large-scale production.

The present disclosure adopts the following technical solution:

According to the present disclosure, the crude theaflavins are used as raw materials, which are dissolved in hot water, allowed to stand still for cooling, and then subjected to suction filtration to remove filter residue, so as to obtain a primary sample solution; the primary sample solution is loaded onto a resin column to enable theaflavins to be adsorbed on resin, and gradient elution is performed with ethanol solutions at different gradients to obtain a primary purified product; after being concentrated by rotary evaporation and freeze-dried, the primary purified product is dissolved in hot water and subjected to suction filtration to remove filter residue, so as to obtain a secondary sample solution; the secondary sample solution is loaded onto a resin column; after theaflavins are adsorbed, gradient elution is performed using ethanol solutions at different gradients to obtain a secondary purified product; and the secondary purified product is concentrated by rotary evaporation and freeze-dried to obtain a high-purity theaflavin product.

The first purpose of the present disclosure is to provide a method for obtaining a high-purity (95% or more) theaflavin product by separating and purifying, and the method specifically includes the following steps:
(1) dissolving crude theaflavins in hot water, letting same stand still for cooling, then carrying out suction filtration to remove filter residue; taking filtrate and loading same onto an LX-20B resin column for chromatography; eluting sequentially with water, an ethanol aqueous solution with a volume ratio of 10%-30% and an ethanol aqueous solution with a volume ratio of 40%-70%; collecting eluent of the ethanol aqueous solution with the volume ratio of 40%-70%, and concentrating by rotary evaporation to obtain a primary purified product; and
(2) mixing the primary purified product with the hot water well, filtering while the mixture is still hot, then taking filtrate and loading same onto an AB-8 resin column for chromatography; eluting sequentially with water, the ethanol aqueous solution with the volume ratio of 10%-30%, an ethanol aqueous solution with a volume ratio of 30%-50% and an ethanol water solution with a volume ratio of 50%-70%; and collecting eluent of the ethanol aqueous solution with the volume ratio of 50%-70%, concentrating by rotary evaporation, and freeze-drying to obtain high-purity theaflavins.

In one embodiment, a purity of theaflavins in the crude theaflavins in step (1) is 15%-30%, and a purity of the high-purity theaflavins in step (2) is not less than 95%.

In one embodiment, in step (1), the crude theaflavins are dissolved in the hot water to obtain a solution with a concentration of 50 mg/mL-200 mg/ml; and a loading flow rate in step (1) is 0.5 BV/h-1.5 BV/h (column bed volume/h); BV-Bed volume; a volume of a chromatographic resin column bed (a part filled with resin).

In one embodiment, in step (2), the primary purified product is mixed well with the hot water to obtain a solution with a concentration of 5 mg/mL-15 mg/ml; and a loading flow rate in step (2) is 0.5 BV/h-1.5 BV/h.

In one embodiment, a height-diameter ratio of the LX-20B resin column in step (1) and/or the AB-8 resin column in step (2) is (10-20): 1.

In one embodiment, in step (1), an elution volume at each gradient is 2 BV-4 BV of water, 4 BV-8 BV of the ethanol aqueous solution with the volume ratio of 10%-30% and 3 BV-6 BV of the ethanol aqueous solution with the volume ratio of 40%-70%, respectively; and an elution flow rate is 1.0 BV/h-2.5 BV/h at each gradient.

In one embodiment, in step (2), an elution volume at each gradient is 2 BV-4 BV of water, 3 BV-6 BV of the ethanol aqueous solution with the volume ratio of 10%-30%, 3 BV-6 BV of the ethanol aqueous solution with the volume ratio of 30%-50%, and 3 BV-6 BV of the ethanol aqueous solution with the volume ratio of 50%-70%, respectively; and an elution flow rate is 1.0 BV/h-2.5 BV/h at each gradient.

In one embodiment, a temperature of the hot water in step (1) and/or step (2) is 60° C.-90° C.

In one embodiment, after step (2), the method further includes:
(3) resin regeneration and recycling: cleaning the LX-20B resin column and the AB-8 resin column using an ethanol aqueous solution with a volume fraction of 95%, and then rinsing same with deionized water until the effluent is free of alcohol odor, the regeneration of the LX-20B resin column also requiring operations: alkaline washing→water washing until neutral→acid washing→water washing until neutral; and repeating steps (1) and (2) for recycling.

In one embodiment, parameters for concentrating by rotary evaporation in step (1) and/or step (2) are the following: a temperature is 40° C.-50° C., and a rotation speed is 70 rpm-100 rpm.

In one embodiment, conditions for freeze-drying are the following: a vacuum degree is 10 Pa-100 Pa, the time is 12 h-24 h, and a temperature is −40° C.-−10° C.

In one embodiment, in step (3), the volume fraction of the ethanol aqueous solution is 95%, the elution flow rate of alkali washing, acid washing and water washing is 1.0 BV/h-2.0 BV/h, the elution volume of the 95% ethanol aqueous solution, alkali washing and acid washing is 2 BV-3 BV, and the elution volume of water washing is 4 BV-6 BV.

In one embodiment, in step (3), an alkaline washing solution is a 2%-4% NaOH solution, and an acid washing solution is a 2%-4% HCl solution.

The second purpose of the present disclosure is to provide a high-purity (95% or more) theaflavin product obtained by separating and purifying with the macroporous resin column coupling method mentioned above.

### Beneficial Effects

According to the method provided by the present disclosure, two specific macroporous resin columns are employed for coupling, and only water and ethanol aqueous solutions are used as eluents to prepare high-purity (95% or more) theaflavins, which can effectively realize the separation of theaflavins from major catechins and caffeine (CAF), and meet demands for high-purity theaflavins in some high-end products and pharmaceutical research and development fields. The method provided by the present disclosure has the following advantages:
(1) The resin used is macroporous adsorption resin, which are relatively low in cost;
(2) the eluents used are only ethanol aqueous solutions, without any other toxic or harmful organic reagents, which is safe and environmentally friendly;
(3) the operation is simple, and the loading quantity of samples is large, so that it is suitable for large-scale production;
(4) macroporous resin is simple in regeneration and thus can be reused for multiple times;
(5) the purity of the obtained theaflavin product is 95% or more, the total recovery rate of theaflavins is greater than 80%, and the yield of 95% high-purity theaflavins is greater than 30%. Therefore, it can be well applied in the separation and purification process of theaflavins, with broad application prospects. Furthermore, it lays a foundation for broadening an application range of theaflavins in the field of high-end products;
(6) the purification method provided by the present disclosure focuses on solving the problems of cumbersome operation, small processing capacity, high cost, and high solvent toxicity in the existing separation and purification technologies, and significantly improves the purity of theaflavins; and
(7) the present disclosure first uses the LX-20B resin column for primary purification, which is conducive to improving loading quantity of samples and purification efficiency and more suitable for the enrichment of low concentration theaflavins compared with other macroporous resins; then, the AB-8 resin column is employed for secondary purification, which can remove the impurities such as caffeine that cannot be removed by the LX-20B resin column, and combines with the specific elution gradients (eluting sequentially with the water, the ethanol aqueous solution with the volume ratio of 10%-30%, the ethanol aqueous solution with the volume ratio of 30%-50% and the ethanol water solution with the volume ratio of 50%-70%); and therefore, the present disclosure is more suitable for the preparation of higher purity theaflavins.

### BRIEF DESCRIPTION OF FIGURES

FIG. 1 shows structures of four major theaflavins; and

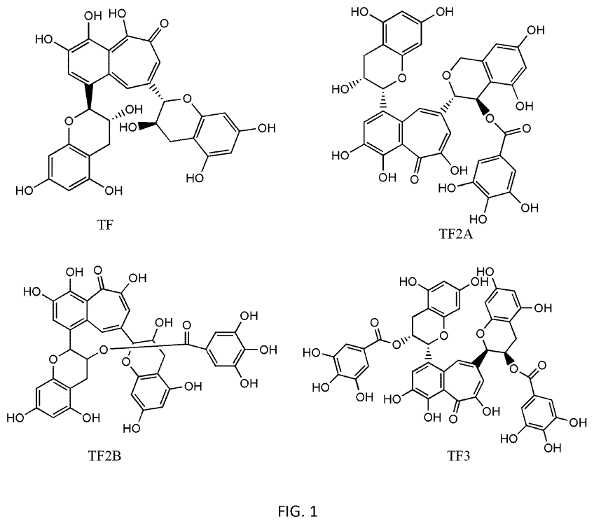

FIG. 2 shows static adsorption/desorption experimental results of theaflavins on different macroporous resin columns in Example 4.

### DETAILED DESCRIPTION

The specific embodiments of the present disclosure will be described in detail with reference to the following examples. The described examples are only some but not all of the examples of the present disclosure, and are intended to explain the present disclosure, rather than to limit the present disclosure.

A determination method involved in the present disclosure:

Determination of the contents of major catechins, caffeine, and four major theaflavins

The components were quantitatively analyzed by high performance liquid chromatography. Quantitative analysis was performed using a high performance liquid chromatograph equipped with a Waters e2695 pump and a Waters PDA detector. The sensitivity of a response value of the method was 0.0001 Au, and determination conditions of the high performance liquid chromatography (HPLC) were as follows: liquid chromatographic column: C₁₈ (particle size 5 μm, 150 mm×4.6 mm); injection volume: 10 μL; column temperature: 30° C.; UV detector: λ=280 nm; mobile phase A: 0.1% (v/v) formic acid/water; mobile phase B: acetonitrile solution. Elution gradients are shown in the table below. Standard curves of 12 reference materials involved are shown in Table 2.

TABLE 1
HPLC gradient elution procedure

| Time/min | 0 | 3 | 7 | 10 | 19 | 20 | 44 | 45 | 49 |
|---|---|---|---|---|---|---|---|---|---|
| Mobile phase A/% | 90 | 90 | 85 | 83 | 83 | 76 | 76 | 90 | 90 |
| Mobile phase B/% | 10 | 10 | 15 | 17 | 17 | 24 | 24 | 10 | 10 |
| Flow rate (mL/min) | 0.6 | 0.6 | 0.6 | 0.6 | 0.6 | 0.8 | 0.8 | 0.6 | 0.6 |

TABLE 2
Standard curves of 12 reference materials

| | | Correlation | Linearity |
|---|---|---|---|
| | | coefficient | range |
| Component | Regression equation | (R²) | (μg/mL) |
| Gallic acid (GA) | y = 36.607x − 59.575 | 0.9972 | 50-500 |
| EGC | y = 1.6374x − 28.997 | 0.9989 | 50-500 |
| Catechin (C) | y = 9.1049x − 114.14 | 0.9997 | 50-500 |
| CAF | y = 26.394x − 18.037 | 0.9974 | 50-500 |
| EC | y = 8.8142x − 54.728 | 0.9997 | 50-500 |
| EGCG | y = 16.726x − 245.57 | 0.9998 | 50-500 |
| Gallocatechin | y = 17.04x − 332.05 | 0.9997 | 50-500 |
| gallate (GCG) | | | |
| ECG | y = 16.959x − 346.28 | 0.9995 | 50-500 |
| TF1 | y = 17.902x − 280.29 | 0.9995 | 50-500 |
| TF2A | y = 15.17x − 432.3 | 0.9995 | 50-500 |
| TF2B | y = 17.764x − 402.05 | 0.9994 | 50-500 |
| TF3 | y = 16.682x − 1040.6 | 0.9990 | 50-500 |

AB-8 resin is a weakly polar copolymer resin of styrene type, which has good adsorption properties for compounds with certain hydrophobicity but with weakly polar groups, and is used for enrichment and purification of target materials. LX-20B resin is a kind of weak polar macroporous adsorption resin of styryl-divinylbenzene skeleton. The resin has high specific surface area, uniform pores, strong selectivity for non-polar and weakly polar molecules in solution system, and has the effect of chromatography separation. Specific parameter information is shown in Table 3 and Table 4:

TABLE 3
LX-20B resin parameters

| Appearance | Opalescent opaque globular |
|---|---|
| | particles |
| polarity | Low polarity |
| Particle size (Particle size range 0.10~0.50 | ≥95 |
| mm), % | |
| Specific surface area, m²/g | ≥800 |
| Mean aperture, A | 27~30 |
| porosity, % | 28-32% |
| Pore volume, ml/g | 1.33-1.35 |
| Maximum service temperature, ° C | 150 |

TABLE 4
AB-8 resin parameters

| Appearance | Opalescent opaque globular |
|---|---|
| | particles |
| polarity | nonpolar |
| Particle size (Particle size range 0.3~1.25 | ≥95 |
| mm), % | |
| Water content, % | 65~75 |
| Wet true density, g/mL | 1.00~1.10 |
| Wet apparent density, g/mL | 0.60~0.70 |
| Specific surface area, m²/g | 480~520 |
| Skeleton density, g/mL | 1.13~1.17 |
| Mean aperture, nm | 13~14 |
| porosity, % | 42~46 |
| Pore volume, ml/g | 0.73~0.77 |
| Maximum service temperature, ° C. | 150 |

### Example 1

(1) Pre-treated LX-20B resin and AB-8 resin were loaded into glass chromatography columns by a wet process according to a height-diameter ratio of 12:1; the specifications of the glass chromatography columns were @25×400 mm (filled with LX-20B internally) and @16×300 mm (filled with AB-8 internally), respectively; and an LX-20B resin column and an AB-8 resin column were obtained;
(2) 10 g of crude theaflavins with a purity of 25% (main impurities and contents: catechins: 28.58±0.05%, caffeine: 1.03±0.06%, protein: 5.27±0.26%, polysaccharide: 5.31±0.58%) were weighed and dissolved in 100 ml of hot water at 70° C. to prepare a 100 mg/ml solution; the obtained solution was allowed to stand still and cooled for 6 h, and then subjected to suction filtration to remove filter residue; and the filtrate was loaded onto the LX-20B resin column through a peristaltic pump at a flow rate of 0.5 BV/h;
(3) after the sample loading was completed, gradient elution was performed using 3 BV of water, 6 BV of a 25% (v/v) ethanol aqueous solution, and 3 BV of a 65% (v/v) ethanol aqueous solution sequentially, with an elution flow rate of 2.0 BV/h; eluent of the 65% (v/v) ethanol aqueous solution was collected and concentrated by rotary evaporation to remove ethanol; and then, the concentrated solution was freeze-dried to obtain a primary purified product. It could be seen from the analysis by high performance liquid chromatography that the purity of theaflavins in the primary purified product was 65.79±0.91%, with a yield of 51.41±1.57% and a total recovery rate of 84.28±2.10%;
(4) 1.00 g of the primary purified product was weighed and dissolved in 100 ml of the hot water at 70° C. to prepare a 10 mg/ml solution; filter residue was removed by filtering while the solution was hot, and the filtrate was loaded onto the AB-8 resin column through a peristaltic pump at a flow rate of 1.0 BV/h;
(5) after the sample loading was completed, gradient elution was performed using 3 BV of water, 6 BV of a 25% (v/v) ethanol aqueous solution, 6 BV of a 35% (v/v) ethanol aqueous solution, and 4 BV of a 50% (v/v) ethanol aqueous solution sequentially, with an elution flow rate of 2.0 BV/h; eluent of the 50% (v/v) ethanol aqueous solution was collected and concentrated by rotary evaporation to remove ethanol; and then, the concentrated solution was freeze-dried to obtain a high-purity theaflavin product. It could be seen from the analysis by high performance liquid chromatography that the purity of theaflavins in the high-purity theaflavin product was 95.41±0.77%, with a yield of 55.34±1.95% and a total recovery rate of 95.83±0.79%;
(6) resin regeneration: the LX-20B resin column and the AB-8 resin column were separately cleaned with 2 BV of a 95% (v/v) ethanol aqueous solution, and then eluted with 4 BV of deionized water until the effluent is free of obvious alcohol odor before stopping; it was necessary to first elute the LX-20B resin column with 2 BV of a 4% NaOH solution and wash same with water until neutral; then the product was eluted with 2 BV of a 2% HCl solution and finally washed with water until neutral, and the above elution flow rate was 1.0 BV/h; and after the resin regeneration treatment was finished, steps (2) to (5) could be repeated for the next round of purification; and
(7) after 5 cycles of regeneration and recycling of the two resins, there was no significant decrease in the purity and yield of the obtained theaflavin product; and after 5 cycles of regeneration and recycling, the obtained high-purity theaflavin product had a theaflavin purity of 95.10±0.69%, a yield of 30.28±1.51%, and a total recovery rate of 80.46±1.01%.

### Example 2

(1) Pre-treated LX-20B resin and AB-8 resin were loaded into glass chromatography columns by a wet process according to a height-diameter ratio of 15:1; the specifications of the glass chromatography columns were @25×500 mm (LX-20B) and @16×300 mm (AB-8), respectively; and an LX-20B resin column and an AB-8 resin column were obtained;
(2) 15 g of crude theaflavins with a purity of 20% (main impurities and contents: catechins: 35.10±0.05%, caffeine: 1.55±0.03%, protein: 6.36±0.35%, polysaccharide: 6.43±0.50%) were weighed and dissolved in 100 ml of hot water at 80° C. to prepare a 150 mg/ml solution; the obtained solution was allowed to stand still and cooled for 12 h, and then subjected to suction filtration to remove filter residue; and the filtrate was loaded onto the LX-20B resin column through a peristaltic pump at a flow rate of 1.0 BV/h;
(3) after the sample loading was completed, gradient elution was performed using 4 BV of water, 6 BV of a 30% (v/v) ethanol aqueous solution, and 3 BV of a 70% (v/v) ethanol aqueous solution sequentially, with an elution flow rate of 1.0 BV/h; eluent of the 70% (v/v) ethanol aqueous solution was collected and concentrated by rotary evaporation to remove ethanol; and then, the concentrated solution was freeze-dried to obtain a primary purified product. It could be seen from the analysis by high performance liquid chromatography that the purity of theaflavins in the primary purified product was 62.48±0.37%, with a yield of 52.98±2.01% and a total recovery rate of 86.19±3.54%;
(4) 1.50 g of the primary purified product was weighed and dissolved in 100 ml of the hot water at 80° C. to prepare a 15 mg/mL solution; filter residue was removed by filtering while the solution was hot, and the filtrate was loaded onto the AB-8 resin column through a peristaltic pump at a flow rate of 1.0 BV/h;
(5) after the sample loading was completed, gradient elution was performed using 3 BV of water, 6 BV of a 20% (v/v) ethanol aqueous solution, 4 BV of a 40% (v/v) ethanol aqueous solution, and 3 BV of a 60% (v/v) ethanol aqueous solution sequentially, with an elution flow rate of 2.0 BV/h; eluent of the 60% (v/v) ethanol aqueous solution was collected and concentrated by rotary evaporation to remove ethanol; and then, the concentrated solution was freeze-dried to obtain a high-purity theaflavin product. It could be seen from the analysis by high performance liquid chromatography that the purity of theaflavins in the high-purity theaflavin product was 95.02±0.39%, with a yield of 58.06±0.23% and a total recovery rate of 95.98±0.11%;
(6) resin regeneration: the LX-20B resin column and the AB-8 resin column were separately cleaned with 2 BV of a 95% (v/v) ethanol aqueous solution, and then eluted with 5 BV of deionized water until the effluent is free of obvious alcohol odor before stopping; it was necessary to first elute the LX-20B resin column with 2 BV of a 4% NaOH solution and wash same with water until neutral; then the product was eluted with 2 BV of a 2% HCl solution and finally washed with water until neutral, and the above elution flow rate was 1.5 BV/h; and after the resin regeneration treatment was finished, steps (2) to (5) could be repeated for the next round of purification; and
(7) after 5 cycles of regeneration and recycling of the two resins, there was no significant decrease in the purity and yield of the obtained theaflavin product; and after 5 cycles of regeneration and recycling, the obtained high-purity theaflavin product had a theaflavin purity of 95.32±0.44%, a yield of 30.49±1.25%, and a total recovery rate of 81.72±1.61%.

### Example 3

(1) Pre-treated AB-8 resin and LX-20B resin were loaded into glass chromatography columns (@16×400 mm) by a wet process according to a height-diameter ratio of 20:1, respectively, to obtain an LX-20B resin column and an AB-8 resin column;
(2) 5 g of crude theaflavins with a purity of 15% (main impurities and contents: catechins: 42.63±0.04%, caffeine: 1.38±0.03%, protein: 6.95±0.40%, polysaccharide: 6.96±0.66%) were weighed and dissolved in 25 ml of hot water at 90° C. to prepare a 200 mg/ml solution; the obtained solution was allowed to stand still and cooled for 8 h, and then subjected to suction filtration to remove filter residue; and the filtrate was loaded onto the LX-20B resin column through a peristaltic pump at a flow rate of 0.5 BV/h;
(3) after the sample loading was completed, gradient elution was performed using 3 BV of water, 8 BV of a 30% (v/v) ethanol aqueous solution, and 6 BV of a 50% (v/v) ethanol aqueous solution sequentially, with an elution flow rate of 2.0 BV/h; eluent of the 50% (v/v) ethanol aqueous solution was collected and concentrated by rotary evaporation to remove ethanol; and then, the concentrated solution was freeze-dried to obtain a primary purified product. It could be seen from the analysis by high performance liquid chromatography that the purity of theaflavins in the primary purified product was 63.14±0.26%, with a yield of 50.95±1.21% and a total recovery rate of 85.33±2.61%;
(4) 0.25 g of the primary purified product was weighed and dissolved in 50 ml of the hot water at 90° C. to prepare a 5 mg/ml solution; filter residue was removed by filtering while the solution was hot, and the filtrate was loaded onto the AB-8 resin column through a peristaltic pump at a flow rate of 1.0 BV/h;
(5) after the sample loading was completed, gradient elution was performed using 3 BV of water, 3 BV of a 30% (v/v) ethanol aqueous solution, 3 BV of a 40% (v/v) ethanol aqueous solution, and 3 BV of a 50% (v/v) ethanol aqueous solution sequentially, with an elution flow rate of 2.0 BV/h; eluent of the 50% (v/v) ethanol aqueous solution was collected and concentrated by rotary evaporation to remove ethanol; and then, the concentrated solution was freeze-dried to obtain a high-purity theaflavin product. It could be seen from the analysis by high performance liquid chromatography that the purity of theaflavins in the high-purity theaflavin product was 95.90±0.59%, with a yield of 57.44±0.81% and a total recovery rate of 94.94±0.85%;
(6) resin regeneration: the LX-20B resin column and the AB-8 resin column were separately cleaned with 2 BV of a 95% (v/v) ethanol aqueous solution, and then eluted with 6 BV of deionized water until the effluent is free of obvious alcohol odor before stopping; it was necessary to first elute the LX-20B resin column with 2 BV of a 4% NaOH solution and wash same with water until neutral; the product was eluted with 2 BV of a 2% HCl solution and finally washed with water until neutral, and the above elution flow rate was 2.0 BV/h; and after the resin regeneration treatment was finished, steps (2) to (5) could be repeated for the next round of purification; and
(7) after 5 cycles of regeneration and recycling of the two resins, there was no significant decrease in the purity and yield of the obtained theaflavin product; and after 5 cycles of regeneration and recycling, the obtained high-purity theaflavin product had a theaflavin purity of 95.11±0.23%, a yield of 30.26±0.97%, and a total recovery rate of 81.09±1.27%.

### Example 4: Screening of Macroporous Resin Columns

Through static adsorption/desorption experiments, the macroporous resin LX-20B with high adsorption/desorption for theaflavins was screened out from six different macroporous resins. The specific experiments were as follows:

1.00 g (dry weight) of pretreated macroporous resin was weighed and placed in a 50 mL conical flask, 20 mL of a solution with a crude theaflavin content of 100 mg/ml was added, the resulting mixture was oscillated for adsorption on a constant temperature shaker at 25° C. for 12 h at 130 r/min, and then the adsorbed saturated resin was filtered to obtain filtrate; the adsorbed saturated resin was cleaned for 2-3 times with deionized water; after washing, the water on the surface of the resin was blotted up using filter paper, 20 ml of a 95% (v/v) ethanol solution was added, and oscillatory desorption was continued on a constant temperature shaker at 25° C. for 12 h at 130 r/min to obtain a desorption solution; and concentrations of theaflavins in the initial solution, filtrate and desorption solution were determined by HPLC. The static equilibrium adsorption capacity (Qₑ), desorption capacity (Q_{d}) and desorption rate (D) of the resin for theaflavins were calculated, and the appropriate resin was selected on this basis. The calculation formula is as follows:

$${Q}_{e} = \frac{( {C}_{0} - {C}_{1} ) \times {V}_{1}}{W}$$

$${Q}_{d} = {C}_{2} \times {V}_{2} / W$$

$$D = \frac{{C}_{2} \times {V}_{2}}{( {C}_{0} - {C}_{1} ) \times {V}_{1}} \times 100 \%$$

In the formula, Qₑ and Q_{d} are respectively the static adsorption capacity and desorption capacity (mg/g dry resin) for theaflavins, D is the desorption rate (%), C₀ is the initial concentration (mg/ml) of theaflavins, C₁ is the equilibrium concentration (mg/ml) of theaflavins, C₂ is the total content (mg/mL) of theaflavins in the desorption solution, V₁ is the volume (mL) of the adsorption solution of theaflavins, V₂ is the volume (mL) of the desorption solution of theaflavins, and W is the dry weight (g) of resin.

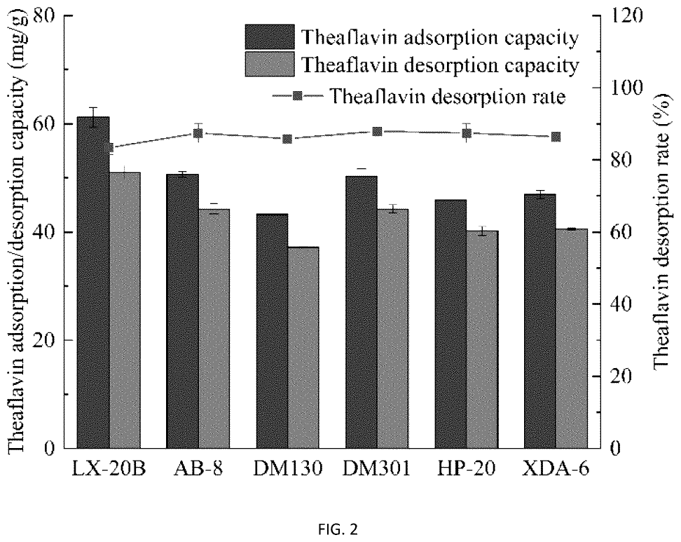

In this example, the exchange capacities of six macroporous resins for four theaflavins were tested, and the suitability of the different macroporous resin for the separation and purification of theaflavins was evaluated by examining their static adsorption/desorption capacities and desorption rates for theaflavins. The results are shown in FIG. 2. The adsorption capacity and desorption capacity of LX-20B resin for theaflavins are significantly higher than those of other resins. Considering that the desorption rates of different resins for theaflavins are not much different and are all 80% or more, LX-20B resin is considered as the first choice for purifying theaflavins.

### Comparative Example 1

Through dynamic elution experiment, AB-8 resin, which was more suitable for this experiment, was selected from macroporous resins suitable for separating and removing caffeine. The specific experiment was as follows:

Compared with Example 3, the difference was only that replacing the AB-8 resin column with the LX-8 resin column to obtain a high-purity theaflavin product; and it could be seen from the analysis by high performance liquid chromatography that the purity of theaflavins in the high-purity theaflavin product was 73.16±1.58%, with a yield of 18.48±0.69% and a total recovery rate of 35.50±0.93%.

Compared with Example 3, the difference of Comparative Example 1 was only in that the resin column used for secondary purification was different. As a result of comparison, it can be found that the purification effect (purity, yield and total recovery rate of theaflavins) of theaflavins by the LX-20B resin coupled with the LX-8 resin under the same conditions is far worse than that by the LX-20B resin coupled with the AB-8 resin used in the present disclosure.

### Comparative Example 2

(1) Pre-treated AB-8 resin and LX-20B resin were loaded into glass chromatography columns (@16×400 mm) by a wet process according to a height-diameter ratio of 20:1, respectively, to obtain an LX-20B resin column and an AB-8 resin column;
(2) 5 g of crude theaflavins with a purity of 15% (main impurities and contents: catechins: 42.63±0.04%, caffeine: 1.38±0.03%, protein: 6.95±0.40%, polysaccharide: 6.96±0.66%) were weighed and dissolved in 25 mL of hot water at 90° C. to prepare a 200 mg/ml solution; the obtained solution was allowed to stand still and cooled for 8 h, and then subjected to suction filtration to remove filter residue; and the filtrate was loaded onto the LX-20B resin column through a peristaltic pump at a flow rate of 0.5 BV/h;
(3) after the sample loading was completed, gradient elution was performed using 3 BV of water, 8 BV of a 10% (v/v) ethanol aqueous solution, and 6 BV of a 35% (v/v) ethanol aqueous solution sequentially, with an elution flow rate of 2.0 BV/h; eluent of the 35% (v/v) ethanol aqueous solution was collected and concentrated by rotary evaporation to remove ethanol; and then, the concentrated solution was freeze-dried to obtain a primary purified product. It could be seen from the analysis by high performance liquid chromatography that the purity of theaflavins in the primary purified product was 23.26±0.59%, with a yield of 32.91±1.28% and a total recovery rate of 37.31±2.09%;
(4) 0.25 g of the primary purified product was weighed and dissolved in 50 ml of the hot water at 90° C. to prepare a 5 mg/mL solution; filter residue was removed by filtering while the solution was hot, and the filtrate was loaded onto the AB-8 resin column through a peristaltic pump at a flow rate of 1.0 BV/h; and
(5) after the sample loading was completed, gradient elution was performed using 3 BV of water, 3 BV of a 30% (v/v) ethanol aqueous solution, 3 BV of a 40% (v/v) ethanol aqueous solution, and 3 BV of a 50% (v/v) ethanol aqueous solution sequentially, with an elution flow rate of 2.0 BV/h; eluent of the 50% (v/v) ethanol aqueous solution was collected and concentrated by rotary evaporation to remove ethanol; and then, the concentrated solution was freeze-dried to obtain a high-purity theaflavin product. It could be seen from the analysis by high performance liquid chromatography that the purity of theaflavins in the high-purity theaflavin product was 68.63±0.81%, with a yield of 56.22±0.93% and a total recovery rate of 94.15±0.96%.

### Comparative Example 3

(1) Pre-treated AB-8 resin and LX-20B resin were loaded into glass chromatography columns (@16×400 mm) by a wet process according to a height-diameter ratio of 20:1, respectively, to obtain an LX-20B resin column and an AB-8 resin column;
(2) 5 g of crude theaflavins with a purity of 15% (main impurities and contents: catechins: 42.63±0.04%, caffeine: 1.38±0.03%, protein: 6.95±0.40%, polysaccharide: 6.96±0.66%) were weighed and dissolved in 25 ml of hot water at 90° C. to prepare a 200 mg/ml solution; the obtained solution was allowed to stand still and cooled for 8 h, and then subjected to suction filtration to remove filter residue; and the filtrate was loaded onto the LX-20B resin column through a peristaltic pump at a flow rate of 0.5 BV/h;
(3) after the sample loading was completed, gradient elution was performed using 3 BV of water, 8 BV of a 30% (v/v) ethanol aqueous solution, and 6 BV of a 50% (v/v) ethanol aqueous solution sequentially, with an elution flow rate of 2.0 BV/h; eluent of the 50% (v/v) ethanol aqueous solution was collected and concentrated by rotary evaporation to remove ethanol; and then, the concentrated solution was freeze-dried to obtain a primary purified product. It could be seen from the analysis by high performance liquid chromatography that the purity of theaflavins in the primary purified product was 63.14±0.26%, with a yield of 50.95±1.21% and a total recovery rate of 85.33±2.61%;
(4) 0.25 g of the primary purified product was weighed and dissolved in 50 ml of the hot water at 90° C. to prepare a 5 mg/ml solution; filter residue was removed by filtering while the solution was hot, and the filtrate was loaded onto the AB-8 resin column through a peristaltic pump at a flow rate of 1.0 BV/h; and
(5) after the sample loading was completed, gradient elution was performed using 3 BV of water, 3 BV of a 10% (v/v) ethanol aqueous solution, 3 BV of a 30% (v/v) ethanol aqueous solution, and 3 BV of a 40% (v/v) ethanol aqueous solution sequentially, with an elution flow rate of 2.0 BV/h; eluent of the 30% (v/v) ethanol aqueous solution was collected and concentrated by rotary evaporation to remove ethanol; and then, the concentrated solution was freeze-dried to obtain a high-purity theaflavin product. It could be seen from the analysis by high performance liquid chromatography that the purity of theaflavins in the high-purity theaflavin product was 42.57±0.78%, with a yield of 37.50±0.99% and a total recovery rate of 40.17±0.72%.

The examples provided above are not intended to limit the scope covered by the present disclosure, nor are the steps described to limit their execution order. Those skilled in the art can make obvious improvements to the present disclosure based on the existing common general knowledge, which also fall within the protection scope defined in the Claims of the present disclosure.

What is claimed is:

1. A method for preparing high-purity theaflavins by coupling macroporous resin columns, comprising the following steps:
(1) dissolving crude theaflavins in hot water, letting same stand still for cooling, then carrying out suction filtration to remove filter residue; taking filtrate and loading same onto an LX-20B resin column for chromatography; eluting sequentially with water, an ethanol aqueous solution with a volume ratio of 10%-30% and an ethanol aqueous solution with a volume ratio of 40%-70%; collecting eluent of the ethanol aqueous solution with the volume ratio of 40%-70%, and concentrating by rotary evaporation to obtain a primary purified product; and
(2) mixing the primary purified product with hot water well, filtering while the mixture is still hot, then taking filtrate and loading same onto an AB-8 resin column for chromatography; eluting sequentially with water, the ethanol aqueous solution with the volume ratio of 10%-30%, an ethanol aqueous solution with a volume ratio of 30%-50% and an ethanol aqueous solution with a volume ratio of 50%-70%; and collecting eluent of the ethanol aqueous solution with the volume ratio of 50%-70%, concentrating by rotary evaporation, and freeze-drying to obtain high-purity theaflavins.

2. The method according to claim 1, wherein a purity of theaflavins in the crude theaflavins in step (1) is 15%-30%, and a purity of the high-purity theaflavins in step (2) is not less than 95%.

3. The method according to claim 1, wherein in step (1), the crude theaflavins are dissolved in the hot water to obtain a solution with a concentration of 50 mg/ml-200 mg/mL; and a loading flow rate in step (1) is 0.5 bed volume/hour (BV/h) to 1.5 BV/h.

4. The method according to claim 1, wherein in step (2), the primary purified product is mixed well with the hot water to obtain a solution with a concentration of 5 mg/mL-15 mg/ml; and a loading flow rate in step (2) is 0.5 bed volume/hour (BV/h) to 1.5 BV/h.

5. The method according to claim 1, wherein a height-diameter ratio of the LX-20B resin column in step (1) and/or the AB-8 resin column in step (2) is (10-20): 1.

6. The method according to claim 1, wherein in step (1), an elution volume at each gradient is 2 bed volume (BV)-4 BV of water, 4 BV-8 BV of the ethanol aqueous solution with the volume ratio of 10%-30% and 3 BV-6 BV of the ethanol aqueous solution with the volume ratio of 40%-70%, respectively; and an elution flow rate is 1.0 BV/h-2.5 BV/h at each gradient.

7. The method according to claim 1, wherein in step (2), an elution volume at each gradient is 2 bed volume (BV)-4 BV of water, 3 BV-6 BV of the ethanol aqueous solution with the volume ratio of 10%-30%, 3 BV-6 BV of the ethanol aqueous solution with the volume ratio of 30%-50%, and 3 BV-6 BV of the ethanol aqueous solution with the volume ratio of 50%-70%, respectively; and an elution flow rate is 1.0 BV/h-2.5 BV/h at each gradient.

8. The method according to claim 1, wherein a temperature of the hot water in step (1) and/or step (2) is 60° C.-90° C.

9. The method according to claim 1, wherein after step (2), the method further comprises:
(3) resin regeneration and recycling: cleaning the LX-20B resin column and the AB-8 resin column using an ethanol aqueous solution with a volume fraction of 95%, and then rinsing same with deionized water until the effluent is free of alcohol odor, the regeneration of the LX-20B resin column also requiring operations: alkaline washing, followed by water washing until neutral, and then acid washing, followed by water washing until neutral; and after these regeneration, steps 1(1) and 1(2) could be repeated for recycling use.

10. The method according to claim 1, wherein parameters for concentrating by rotary evaporation in step (1) and/or step (2) are the following: a temperature is 40° C.-50° C., and a rotation speed is 70 rpm-100 rpm.
